(19) United States
(12) Patent Application Publication
(10) Pub. No.: US 2026/0083629 A1
(43) Pub. Date: Mar. 26, 2026

(54) Body-Worn Therapeutic Speaker Device

(71) Applicant: Susan Low, Rancho Santa Fe, CA (US)

(72) Inventor: Susan Low, Rancho Santa Fe, CA (US)

(21) Appl. No.: 19/338,459

(22) Filed: Sep. 24, 2025

Related U.S. Application Data
(60) Provisional application No. 63/698,070, filed on Sep. 24, 2024.

(51) Int. Cl. A61H 23/02; A61M 21/00; A61M 21/02

(52) U.S. Cl. CPC A61H 23/0236; A61M 21/02; A61H 2201/1207; A61H 2201/1645; A61H 2201/165; A61H 2201/169; A61H 2201/5007; A61M 2021/0022; A61M 2021/0027; A61M 2205/0216; A61M 2205/0266; A61M 2205/3592; A61M 2205/502; A61M 2205/8206; A61M 2205/8237; A61M 2205/8262; A61M 2209/088

(57) ABSTRACT

A body-worn therapeutic speaker device is provided. The device is a body-worn therapeutic speaker designed to deliver vibrational sound therapy and music through wireless-enabled speaker units placed on various areas of the body. The device comprises at least one wireless speaker encased in a deformable, skin-safe material that conforms to different body regions for secure and effective placement. The speaker transmits specific vibrational frequencies, music, and sounds to promote circulation, muscle relaxation, pain relief, and emotional well-being by stimulating cellular activity and regulating neurotransmitter release. The device also includes an electromagnetic prevention mechanism to minimize radiation exposure, a rechargeable or disposable battery for power, and a mobile application for remote control of music, volume, and frequency selection.

### CROSS-REFERENCE TO RELATED APPLICATION

The present application claims priority to, and the benefit of, U.S. Provisional Application No. 63/698,070, which was filed on Sep. 24, 2024, and is incorporated herein by reference in its entirety.

### FIELD OF THE INVENTION

The present invention relates generally to the field of speakers. More specifically, the present invention relates to a body-worn therapeutic speaker that delivers vibrational sound therapy through wireless-enabled speaker units placed on various body areas, using specific frequencies to promote circulation, muscle relaxation, pain relief, and emotional well-being. Accordingly, the present disclosure makes specific reference thereto. Nonetheless, it is to be appreciated that aspects of the present invention are also equally applicable to other like applications, devices, and methods of manufacture.

### BACKGROUND

Many individuals experience aches, tension, and discomfort throughout their bodies due to various factors such as stress, physical strain, and medical conditions. Traditional methods of relief, including massages, medications, and physical therapy, can be effective but may not be suitable for all individuals or may require frequent and costly treatments. Additionally, many people seek holistic alternatives that do not rely on pharmaceutical interventions or invasive procedures. Pain and discomfort in areas such as the back, shoulders, neck, and other muscle groups can significantly impact daily activities and overall well-being. The human body's physiological responses to sound and vibration have been studied for their potential therapeutic benefits, particularly in promoting relaxation, improving circulation, and reducing muscle tension. However, existing therapeutic devices often focus on mechanical massage or electrical stimulation, lacking integration with sound-based therapies. There is a need for a non-invasive, customizable solution that utilizes sound frequencies to provide targeted relief. Such a device could offer a user-friendly and adaptable approach to addressing pain, stress, and muscle tension without requiring professional intervention or repeated expenditures.

Therefore, there exists a long-felt need in the art for Bluetooth body speakers that deliver music, sound, and vibrational frequencies directly to body tissues for therapeutic purposes. There also exists a long-felt need in the art for a body-worn device that is deformable, form-fitting, and comfortable for placement on various areas of the body, allowing for optimal positioning and effective treatment. Moreover, there exists a long-felt need in the art for a non-invasive alternative to conventional pain and anxiety relief methods, offering users a drug-free and holistic approach to managing discomfort and stress.

The subject matter disclosed and claimed herein, in one embodiment thereof, comprises a body-worn therapeutic speaker device. The device is a body-worn therapeutic speaker designed to deliver vibrational sound therapy through wireless-enabled speaker units placed on various areas of the body. The device comprises at least one wireless speaker encased in a deformable, skin-safe material that conforms to different body regions for secure and effective placement. The speaker transmits specific vibrational frequencies to promote circulation, muscle relaxation, pain relief, and emotional well-being by stimulating cellular activity and regulating neurotransmitter release. The device also includes an electromagnetic prevention mechanism to minimize radiation exposure, a rechargeable or disposable battery for power, and a mobile application for remote control of volume and frequency selection. A method of using the device involves positioning the speaker on a targeted body area, wirelessly connecting it to a smart device, selecting desired vibrational frequencies via the mobile application, and initiating a therapy session to facilitate wellness benefits.

In this manner, the body-worn therapeutic speaker device of the present invention accomplishes all the foregoing objectives and provides a wearable therapeutic device that transmits sound waves directly into the user's body. The device can conform to different body areas while allowing users to select and control specific sound frequencies via a mobile application. By delivering vibrational frequencies into body tissues, the device promotes circulation, alleviates muscle tension, reduces pain, and supports relaxation. By leveraging sound-based therapy as an alternative to conventional treatment methods, the device provides an innovative, non-invasive solution for pain and anxiety relief, enhancing overall well-being.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a body-worn therapeutic speaker device. The device is a body-worn therapeutic speaker designed to deliver vibrational frequencies through wireless-enabled speaker units placed on various areas of the body. By projecting music directly into the body, the device facilitates blood flow, meridian stimulation, and cellular resonance, promoting circulation, muscle relaxation, pain relief, stress reduction, and overall well-being.

Each speaker unit includes at least one wireless speaker that connects to a smart device via Wi-Fi, Bluetooth, or other wireless communication methods. The speakers vary in size and placement to target specific therapeutic areas. To ensure safety and minimize electromagnetic interference, each speaker is protected by an electromagnetic field (EMF) protection device. The speaker is encased in a deformable body, allowing it to conform to different anatomical regions such as the chest, back, shoulders, neck, hands, and feet.

Once positioned, the speaker transmits sound waves through the skin, soft tissues, and water content of the body. The vibrational energy facilitates physiological responses, including improved circulation, muscle relaxation, and pain reduction. Therapeutic frequencies emitted by the speaker may also aid in reducing anxiety and depression symptoms by regulating nervous system activity and promoting neurotransmitter release. The speaker supports various vibrational frequencies, music, and sounds including 226 Hz, 359 Hz, 432 Hz, 639 Hz, and 963 Hz, allowing users to customize their therapeutic experience. The speaker is powered by at least one battery, which may be disposable or rechargeable. Embodiments utilizing a rechargeable battery include at least one charging port, such as a USB or wireless charging port.

The device also features a mobile application that provides remote control over speaker functionalities. The application allows users to adjust volume levels and select vibrational frequencies based on personal preferences or therapeutic goals.

A method of using the device includes providing the body-worn therapeutic speaker, positioning it on a desired body area, powering on the wireless speaker, pairing it with a smart device via a wireless connection, and accessing the mobile application to adjust volume and select therapeutic frequencies. During a therapy session, the speaker transmits sound waves, promoting circulation, muscle relaxation, pain relief, stress reduction, and mood enhancement.

Accordingly, the body-worn therapeutic speaker device of the present invention is particularly advantageous as it provides a wearable therapeutic device that transmits sound waves directly into the user's body. The device can conform to different body areas while allowing users to select and control specific sound frequencies via a mobile application. By delivering vibrational frequencies into body tissues, the device promotes circulation, alleviates muscle tension, reduces pain, and supports relaxation. By leveraging sound-based therapy as an alternative to conventional treatment methods, the device provides an innovative, non-invasive solution for pain and anxiety relief, enhancing overall well-being.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a body-worn therapeutic speaker device of the present invention designed for neck use in accordance with the disclosed architecture;

FIG. 2 illustrates a front perspective view of one potential embodiment of a body-worn therapeutic speaker device of the present invention designed for shoulder use in accordance with the disclosed architecture;

FIG. 3 illustrates a rear perspective view of one potential embodiment of a body-worn therapeutic speaker device of the present invention designed for shoulder use and while worn by a user in accordance with the disclosed architecture;

FIG. 4 illustrates multiple perspective views of one potential embodiment of a body-worn therapeutic speaker device of the present invention designed for hand use and while worn around a user's hand in accordance with the disclosed architecture;

FIG. 5 illustrates a schematic view of features of a mobile application of one potential embodiment of a body-worn therapeutic speaker device of the present invention in accordance with the disclosed architecture;

FIG. 6 illustrates a flowchart of a method of using one potential embodiment of a body-worn therapeutic speaker device of the present invention in accordance with the disclosed architecture; and

FIG. 7 illustrates a front view of a garment and a mat of one potential embodiment of a body-worn therapeutic speaker device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for Bluetooth body speakers that deliver music, sound, and vibrational frequencies directly to body tissues for therapeutic purposes. There also exists a long-felt need in the art for a body-worn device that is deformable, form-fitting, and comfortable for placement on various areas of the body, allowing for optimal positioning and effective treatment. Moreover, there exists a long-felt need in the art for a non-invasive alternative to conventional pain and anxiety relief methods, offering users a drug-free and holistic approach to managing discomfort and stress.

The present invention, in one exemplary embodiment, is comprised of a body-worn therapeutic speaker device. The device is a body-worn therapeutic speaker designed to deliver vibrational frequencies through wireless-enabled speaker units positioned on various areas of the body. By projecting music directly into the body, the device supports blood flow, meridian stimulation, and cellular resonance, promoting circulation, muscle relaxation, pain relief, stress reduction, and overall well-being.

Each speaker unit comprises at least one wireless speaker that connects to a smart device via Wi-Fi, Bluetooth, or other wireless communication methods. The speakers vary in size and placement to target specific therapeutic areas. To ensure safety and minimize electromagnetic interference, each speaker is protected by an electromagnetic field (EMF) protection device. The speaker is housed in a deformable body that conforms to different anatomical regions, including the chest, back, shoulders, neck, hands, and feet.

Once positioned, the speaker transmits sound waves through the skin, soft tissues, and water content of the body. The vibrational energy facilitates physiological responses such as improved circulation, muscle relaxation, and pain reduction. Therapeutic frequencies emitted by the speaker may also help alleviate symptoms of anxiety and depression by regulating nervous system activity and promoting neurotransmitter release. The speaker supports various vibrational frequencies, including 226 Hz, 359 Hz, 432 Hz, 639 Hz, and 963 Hz, allowing users to customize their therapeutic experience. The speaker is powered by at least one battery, which may be disposable or rechargeable. Embodiments utilizing a rechargeable battery include at least one charging port, such as a USB or wireless charging port.

The device also incorporates a mobile application that enables remote control over speaker functionalities. The application allows users to adjust volume levels and select vibrational frequencies based on personal preferences or therapeutic needs.

A method of using the device includes providing the body-worn therapeutic speaker, positioning it on a desired body area, powering on the wireless speaker, pairing it with a smart device via a wireless connection, and accessing the mobile application to adjust volume and select therapeutic frequencies, music, and sounds. During a therapy session, the speaker transmits sound waves that promote circulation, muscle relaxation, pain relief, stress reduction, and mood enhancement.

The body-worn therapeutic speaker device provides a wearable solution for transmitting sound waves directly into the user's body. Designed to conform to different body areas, the device allows users to select and control specific sound frequencies via a mobile application. By delivering vibrational frequencies into body tissues, the device promotes circulation, reduces muscle tension, alleviates pain, and enhances relaxation. Leveraging sound-based therapy as an alternative to conventional treatment methods, the device offers a non-invasive solution for pain and anxiety relief, supporting overall well-being.

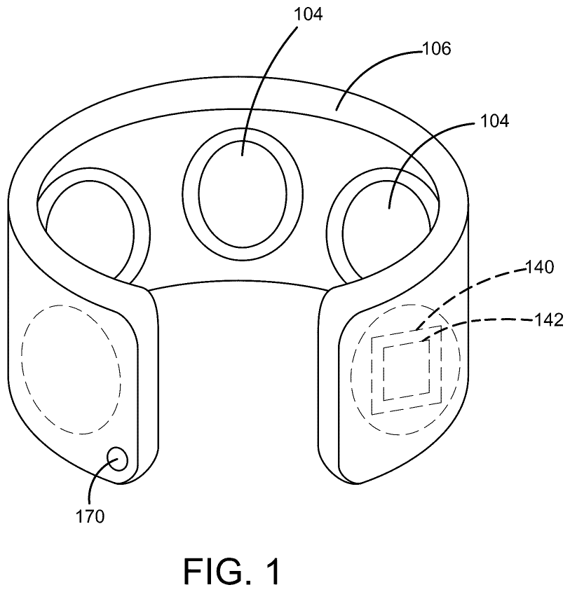

Referring initially to the drawings, FIG. 1 illustrates a front perspective view of one potential embodiment of a body-worn therapeutic speaker device 100 of the present invention designed for neck use in accordance with the disclosed architecture. The device 100 is comprised of a body-worn therapeutic speaker designed to deliver vibrational frequencies through wireless-enabled speaker units 102 that can be placed on various areas of the body. By projecting music directly into a user's body, the device 100 facilitates movement in blood flow, meridians, and cell tissue, bringing the body-mind system into resonance and coherence. The transmission of sound waves through bodily tissues and water content is intended to promote circulation, relax muscles, reduce physical pain, alleviate stress and anxiety, and enhance overall well-being.

Each speaker unit 102 is comprised of at least one wireless speaker 104 that can be wirelessly connected to a smart device via Wi-Fi, Bluetooth, or other wireless communication method. The speaker 104 is configured to vary in size and position on the body 106 across different embodiments, allowing customized placement to target specific areas requiring therapeutic sound waves. To ensure safety and minimize electromagnetic interference, each speaker 104 is protected by an electromagnetic field (EMF) protection device 112. The EMF protection device 112 may be comprised of, but is not limited to, ferrite beads, conductive shielding enclosures, electromagnetic-absorbing materials, or multilayer prevention films designed to reduce exposure to electromagnetic radiation. The speaker 104 may be any type known in the art such as but not limited to a car speaker, a wireless speaker, etc.

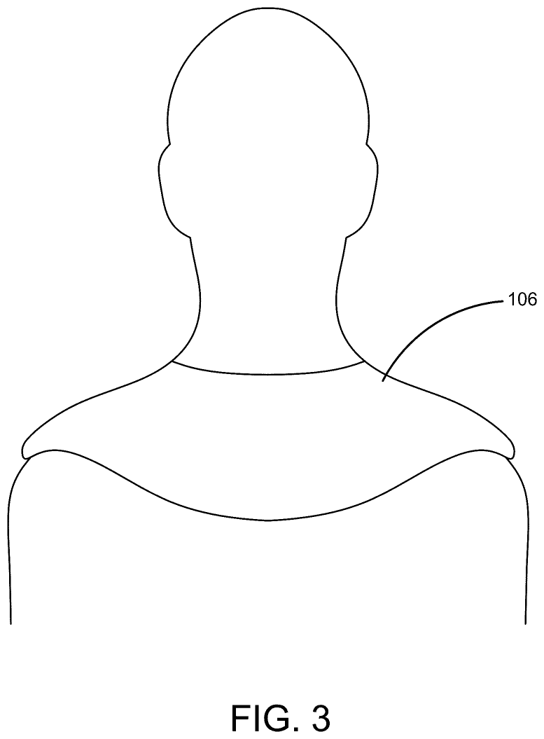

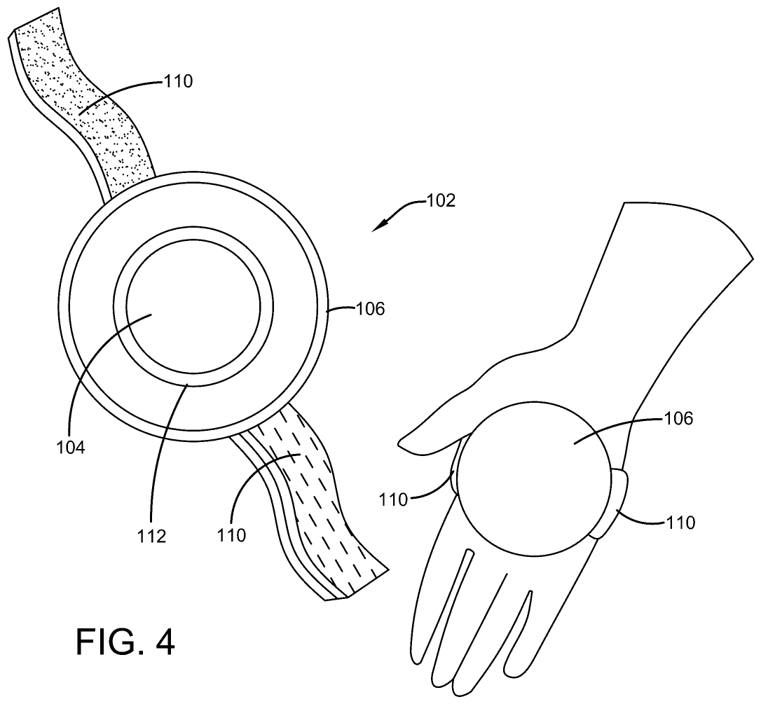

The speaker 104 is encased in a deformable body 106. The body 106 is preferably made from a latex, neoprene, memory foam, or silicone-infused material, though other flexible and skin-safe materials may also be used in different embodiments. The body 106 is further preferably a weighted material. The deformable nature of the body 106 allows it to conform to various body contours while maintaining a secure, gently weighted fit on and around different anatomical regions, such as but not limited to the chest, back, shoulders (as seen in FIG. 3), neck, hands (as seen in FIG. 4), feet, head, and other targeted areas. The body 106 may be configured in multiple shapes and forms to optimize placement and function based on specific therapeutic needs.

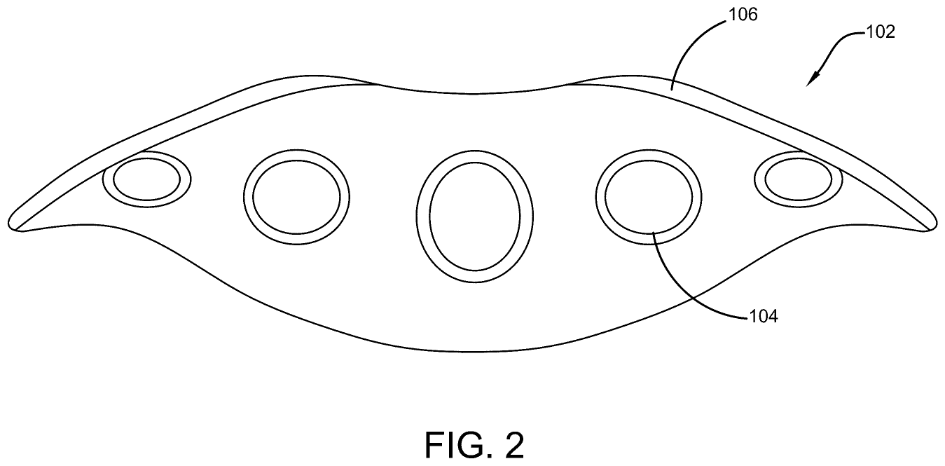

For example, a circular or oval configuration of the body 106 is designed for placement on broader areas such as the chest, back, abdomen, hips, and stomach, ensuring proper contouring to the body's natural shape. A neck configuration of the body 106, as seen in FIG. 1) may be comprised of a memory foam material, allowing for enhanced comfort and ease of application. This configuration enables the body 106 to wrap securely around the neck and provide direct vibration to the cervical and upper trapezius muscles. A shoulder configuration of the body 106 (as seen in FIG. 2 and FIG. 3) is designed to rest below the neck and extend around the shoulders, delivering sound therapy to the trapezius, rhomboid minor, and levator scapulae muscles for deep-tissue vibrational effects. A hand and foot configuration of the body 106 includes a fastener 110, which may be a hook-and-loop strap fastener, a magnetic strap fastener, or another adjustable securing mechanism, ensuring the speaker unit 102 remains in place.

Once placed on or around a user's body, the speaker 104 transmits sound waves directly through the skin, soft tissues, and water content of the body 106. The vibrational energy of the sound waves is designed to facilitate physiological responses such as increased blood circulation, muscular relaxation, and pain reduction. Additionally, the therapeutic frequencies, music, and/or sounds emitted by the speaker 104 may aid in reducing symptoms of anxiety and depression by regulating nervous system activity and promoting the natural release of neurotransmitters such as serotonin, dopamine, oxytocin, and endorphins. The ability of sound waves to resonate with and stimulate cellular activity may further contribute to holistic wellness, encouraging a positive emotional state and enhancing creative cognition. The speaker 104 supports a range of vibrational frequencies (of any level), sounds, low frequencies, and music types including, but not limited to, 226 Hz, 359 Hz, 432 Hz, 639 Hz, and 963 Hz, allowing users to customize their therapeutic experience by selecting specific sound frequencies known for their potential wellness benefits.

The speaker 104 is powered by at least one battery 140, which may be either disposable or rechargeable, depending on the embodiment. In embodiments utilizing a rechargeable battery 140, the device 100 is further comprised of at least one charging port 142, such as, but not limited to, a USB port, a wireless charging port, etc.

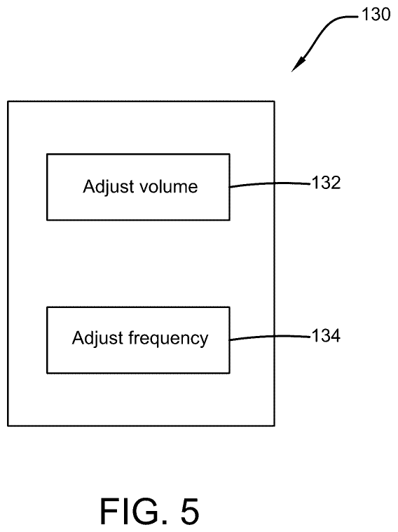

The device 100 is also comprised of a mobile application 130, which provides users with remote control over various functionalities of the speaker 104, as seen in FIG. 5. The application 130 includes a plurality of features designed to enhance user experience and customization. One primary feature allows the user to adjust a volume 132 of the speaker 104, enabling control over sound intensity for different therapeutic settings. In one embodiment, the speaker unit 102 itself is comprised of a physical volume control 170 of any style, type, and configuration. Another feature allows the user to select a sound/music 134 from a music library of the mobile application 130 (or other external audio source) that may have a specific frequency and that is played from the speaker 104, providing the ability to choose between different vibrational frequencies, music, sounds, etc. based on personal preference or specific therapeutic goals. As will be explained more fully below, the mobile application 130 may also allow a user to customize the behavior 135 of the light source 150. The mobile application 130 may also allow a user to select a soundtrack of sound 136 played from the speaker unit 102. The application 130 may have a number of preset protocols/configurations 137 for sleep, creativity, stress relief, etc. The application 130 may also have health tracking capabilities 138 that allows a user to track wellness metrics such as but not limited to biomarkers.

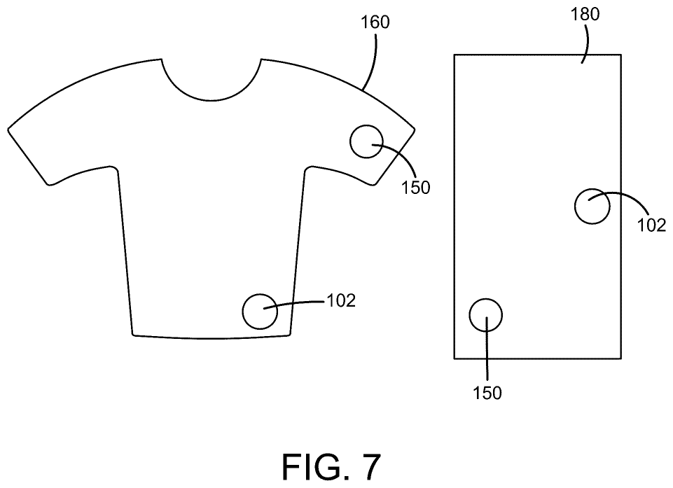

In one embodiment, the speaker unit 102 (and any other components of the device 100 as mentioned) may be positioned on a garment 160 of any style, type, material, and configuration such as but not limited to vests, belts, headbands, body-suits, etc. In one embodiment, the light source 150 is further present on the garment 160, as seen in FIG. 7, wherein the light source may be but is not limited to LED meridian lighting.

In a different embodiment, the device 100 is comprised of a mat 180 that may also integrate any other components of the device 100 as previously mentioned, as seen in FIG. 7.

In one embodiment, the speaker unit 102 may be waterproof and used in water in a non-body-worn capacity such as but not limited to pools, tubs, and Jacuzzis. In this embodiment, the speaker unit 102 is a non-bouyant, weighted speaker such that the speaker unit 102 rests at the bottom of pools, tubs, jacuzzis, etc.

It should be appreciated that in any embodiment the speaker unit 102 may emit any type of sound such as but not limited to frequencies, music, ambient music, dynamic music, etc.

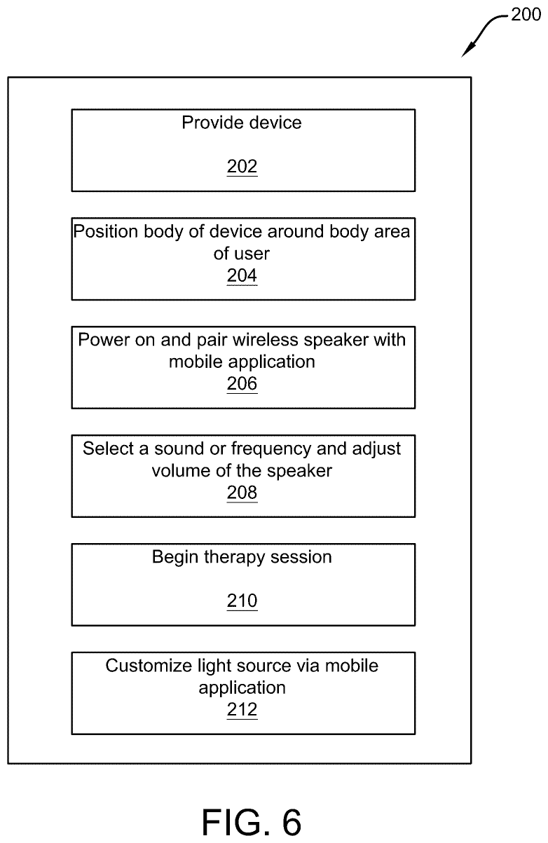

The present invention is also comprised of a method of using 200 the device 100, as seen in FIG. 6. First, a device 100 is provided, comprised of a body-worn therapeutic speaker having at least one speaker unit 102, at least one deformable body 106, at least one wireless speaker 104, and at least one battery 140 [Step 202]. Then, the deformable body 106 is positioned on/around and/or worn on a desired area of a user's body, such as the chest, back, shoulders, neck, hands, or feet, ensuring a secure and comfortable fit [Step 204]. Next, the wireless speaker 104 is powered on, and the device 100 is paired with a smart device via Wi-Fi, Bluetooth, or another wireless communication method [Step 206]. Once connected, a mobile application 130 is accessed on the smart device, allowing the user to adjust a volume 132, select a sound, and/or select a frequency 134 of the speaker 104, choosing from a range of therapeutic frequencies such as 226 Hz, 359 Hz, 432 Hz, 639 Hz, and 963 Hz [Step 208]. Then, the user begins a therapy session, during which the speaker 104 transmits sound waves through the skin, soft tissues, and water content of the body 106, promoting circulation, muscular relaxation, pain relief, stress reduction, and mood enhancement [Step 210]. If applicable, a user can also customize the light source 150 via the mobile application 130 [Step 212].

In one embodiment, the device 100 is comprised of at least one light source 150. The light source is preferably but not limited to a low-voltage (or any voltage type) LED light that may or may not align with the audio output of the speaker unit 102. In one embodiment, the light source 150 may respond to frequency changes, pulse with biofeedback inputs such as but not limited to heart rate, breath rhythm, etc. and/or may also pulse or stream, all of which can be customized by the mobile application 130. In other embodiments, the light source 150 may be turned off or may illuminate constantly.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “body-worn therapeutic speaker device” and “device” are interchangeable and refer to the body-worn therapeutic speaker device 100 of the present invention.

Notwithstanding the foregoing, the body-worn therapeutic speaker device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the body-worn therapeutic speaker device 100 as shown in the FIGS. are for illustrative purposes only, and that many other sizes and shapes of the body-worn therapeutic speaker device 100 are well within the scope of the present disclosure. Although the dimensions of the body-worn therapeutic speaker device 100 are important design parameters for user convenience, the body-worn therapeutic speaker device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A body-worn therapeutic speaker device comprising:
a deformable body configured to conform to a user's body;
a wireless speaker positioned within the body, the wireless speaker configured to transmit a vibrational sound wave;
an electromagnetic field prevention device surrounding the wireless speaker; and
a battery configured to power the wireless speaker.

2. The body-worn therapeutic speaker device of claim 1, wherein the wireless speaker produces a sound, a music, or a vibrational frequency that is 226 Hz, 359 Hz, 432 Hz, 639 Hz, or 963 Hz.

3. The body-worn therapeutic speaker device of claim 1, wherein the electromagnetic field protection device comprises a ferrite bead.

4. The body-worn therapeutic speaker device of claim 1, wherein the electromagnetic field protection device comprises a conductive shielding enclosure.

5. The body-worn therapeutic speaker device of claim 1, wherein the electromagnetic field protection device comprises an electromagnetic-absorbing material.

6. The body-worn therapeutic speaker device of claim 1, wherein the electromagnetic field protection device comprises a multilayer prevention film.

7. The body-worn therapeutic speaker device of claim 1, wherein the battery is comprised of a charging port.

8. The body-worn therapeutic speaker device of claim 7, wherein the charging port is comprised of a wireless charging port.

9. The body-worn therapeutic speaker device of claim 7, wherein the charging port is comprised of a USB port.

10. A body-worn therapeutic speaker device comprising:
a mobile application;
a deformable body configured to conform to a user's body, the deformable body comprised of a fastener;
a wireless speaker positioned within the body, the wireless speaker configured to transmit a vibrational sound wave;
an electromagnetic field protection device surrounding the wireless speaker; and
a battery configured to power the wireless speaker.

11. The body-worn therapeutic speaker device of claim 10, wherein the fastener is comprised of a hook and loop strap.

12. The body-worn therapeutic speaker device of claim 10, wherein the fastener is comprised of a magnetic strap.

13. The body-worn therapeutic speaker device of claim 10, wherein the mobile application is configured to allow a user to adjust a volume of the wireless speaker.

14. The body-worn therapeutic speaker device of claim 10, wherein the mobile application is configured to allow a user to adjust a sound or a music played from the wireless speaker.

15. The body-worn therapeutic speaker device of claim 10, wherein the charging port is comprised of a wireless charging port.

16. The body-worn therapeutic speaker device of claim 10, wherein the charging port is comprised of a USB port.

17. The body-worn therapeutic speaker device of claim 10, wherein the wireless speaker produces a sound, a music, or a vibrational frequency that is 226 Hz, 359 Hz, 432 Hz, 639 Hz, or 963 Hz.

18. The body-worn therapeutic speaker device of claim 10, wherein the deformable body is comprised of a silicone material.

19. The body-worn therapeutic speaker device of claim 10, wherein the deformable body is comprised of a memory foam material.

20. A method of using a body-worn therapeutic speaker device, the method comprising the following steps:
providing a body-worn therapeutic speaker device comprised of a speaker unit, a deformable body, a wireless speaker, and a battery;
positioning the deformable body on a user's body area;
powering on the wireless speaker;
pairing the device with a smart device via a wireless communication method;
accessing a mobile application on the smart device;
adjusting a volume and selecting an audio type played by the wireless speaker via the mobile application; and
transmitting sound waves from the wireless speaker through the deformable body to the user's body.
